(19) United States
(12) Patent Application Publication
(10) Pub. No.: US 2004/0249448 A1
(43) Pub. Date: Dec. 9, 2004

(54) Device for active phase cell or tissue sampling and uses thereof

(76) Inventor: Philippe Gault, Orleans (FR)

Correspondence Address: NIXON & VANDERHYE, PC, 1100 N GLEBE ROAD, 8TH FLOOR, ARLINGTON, VA, 22201-4714, US

(21) Appl. No.: 10/498,122

(22) PCT Filed: Dec. 17, 2002
(86) PCT No.: PCT/FR02/04410

(30) Foreign Application Priority Data
Dec. 19, 2001 (FR) 01 16456

(51) Int. Cl. A61F 2/06

(52) U.S. Cl. 623/1.41; 623/13.17; 623/23.63; 623/23.72

(57) ABSTRACT

The invention concerns a device and methods for providing implants, cells or tissues, for example for grafts or in vitro research cultures, of tissue engineering or cellular therapy. The invention is particularly adapted to produce, in sufficient amount, specific cell populations with high proliferation and/or differentiation potential, adapted for use in tissue engineering, in repair surgery or therapy, particularly for humans. The invention also concerns a method for preparing such a device and its uses, in particular for treating tissue loss, and kits, tranplants, tissues or biological preparations.

The present invention concerns a device and methods for providing implants, cells or tissues, for example for grafts or in vitro research cultures, of tissue engineering or cellular therapy. The invention is particularly adapted to produce, in sufficient amount, specific cell populations, with high proliferation and/or differentiation potential adapted for use in tissue engineering, in repair surgery or therapy, particularly for humans. The invention also concerns a method for preparing such a device and its uses, in particular for treating tissue loss, and kits, transplants, tissues or biological preparations.

The use of mammalian cells for experimental research (genetic studies, toxicity studies, and the like) and for pharmacological or therapeutic purposes (cellular therapy, tissue repair, and the like) has become increasingly widespread. To this end, different methods for obtaining cells or tissues have been developed, in particular for preparing primary cell cultures, based principally on sampling and ex vivo treatment of a biopsy. In general, conventional biopsy techniques are characterized by the fact that the samples are taken from mature tissues. Now, the latter contain large amounts of intercellular matrix and often, few cells. Moreover, the cells contained in the samples are for the most part quiescent. Such cells are therefore not in optimal physiological conditions for uses in tissue repair.

The present invention provides a novel approach for preparing cells, transplants, tissues and, more generally, biological material comprising mammalian cells, particularly in view of their implantation in vivo or their use for research experiments or industrial or pharmacogenomic screening.

More particularly, the present invention provides for the recovery of cells activated by a controlled surgical trauma followed by a healing period, and methods which facilitate said recovery in a surprising manner.

In particular, the invention has as its object a device for providing implants, cells or tissues. More specifically, the invention concerns a device, the use of which is preferably limited in time, such as defined hereinabove comprising a wall made of biocompatible material defining a hollow space, said wall containing pores allowing cells to colonize said hollow space.

The invention also concerns a method for preparing an implant, tissue or cell culture, comprising (i) introducing a device such as defined hereinabove in the tissue of a subject, (ii) maintaining the device for a sufficient period of time to allow colonization of said device by cells displaying the desired properties and (iii) removing the device and recovering the cells, which may be placed in culture, stored, packaged and/or grafted.

Thus, the invention concerns a device such as defined hereinabove for preparing an implant, tissue or cell culture.

The invention further concerns the use of a device according to the invention for preparing a composition for carrying out a method of therapeutic or surgical treatment of the human body, in particular a cell therapy method.

The invention also concerns the use of a device according to the invention for preparing a kit for in vivo sampling of cells and/or tissue in a subject.

The invention also relates to a kit containing a device such as described hereinabove. It further concerns surgical instruments, generally single use, associated with each type of device which may also be presented in the form of kits.

The invention also relates to a cell culture, cell preparation, implant or tissue comprising cells, obtained by a hereinabove method.

The device and the methods of the invention promote cell proliferation and differentiation, extracellular matrix production, and tissue organization and maturation by allowing an optimal culture carried out under natural physiological conditions. Thus the tissue surrounding the site of implantation of the device ensures homogeneous and suitable nutrition and therefore growth, itself homogeneous, of the tissue to be reconstituted proliferating inside the device.

Description of the Device

As indicated, the invention is based on a novel approach for preparing biological material. In particular, the invention is based on methods and devices allowing recovery in vivo of active phase cells from tissues of interest. The invention thus describes a specific device, implantable in vivo, by which to trap cells of interest. The inventive device further provides a space for growth for the cells, which is not crushed or made smaller by pressure from surrounding tissues. Said device also allows easy recovery of the tissue and cells which have proliferated inside it. The use of said device is therefore preferably limited in time, since, after recovery, it is removed from the tissue(s) wherein it was placed.

More particularly, the inventive device comprises a wall made of biocompatible material defining a hollow space, said wall containing pores allowing cells to colonize said hollow space.

Said pores advantageously have a sufficient area such that the permeability of the wall of the device allows rapid colonization. Their area must not be too large so as to avoid excess retention in tissues or difficulty in extracting the device from the tissue. In fact, removal may cause a laceration of the tissue embedded in the internal space of the device and the loss of a part of said tissue. The ratio between the total area of the pores and the overall external area of the device wall may vary according to the tissue and the desired stage of healing. Said ratio is preferably less than 0.5, in particular comprised between 0.1 and 0.5, typically between 0.1 and 0.4. A ratio of 0.2 is preferred for bone and ligament tissue for example.

The pores present in the wall of the device may have a variety of forms, sizes and arrangements.

While round pores may be suited to some tissues, it may also be of interest to choose elongated or oval pores for easier removal of the device from a fibrous tissue, such as a ligament for example, by positioning the main axis of the pores perpendicular to the fibers. The invention therefore provides devices the perforations of which may be round, elongated and/or oval and/or any other form suited to the tissue of interest, in particular any other form facilitating the colonization of cells, the regeneration of the tissue and/or the implantation or removal of the trap. A same device may contain pores of essentially the same size and regularly arranged. However, in general, it is possible to produce devices containing pores of different forms and/or different sizes and/or irregularly arranged on the wall.

The wall of the device may be supple or more or less rigid according to the tissue to be sampled. The material used to produce the device must be biocompatible so that is does not negatively interfere with cell proliferation. In so far as the device is not an implant, that is to say not designed to integrate within a tissue, but intended to provide cells, implants or tissues, it is not necessary for the material used to be selected for its resistance to pressure such as would be the case, for instance, of the material used to prepare an implant to replace a vertebral disk or a part of the spinal column. Titanium, titanium alloys, ceramics, chrome-cobalt, nickel-chrome alloys, polymers such as polytetrafluoroethane or polycarbonate may be used, for example, although this list is not exhaustive.

According to a particular embodiment, the wall of the inventive device is therefore made in a biocompatible material based on titanium, titanium alloys, ceramics, chrome-cobalt, nickel-chrome alloys, or polymers such as polytetrafluoroethane or polycarbonate.

The inventive device may adopt a variety of external forms, according to the tissue from which one wants to recover cells. Said form is defined by the wall of the device and may for example be that of a cylinder (particularly for bone tissue), a spindle (particularly for ligaments), a trunk-cone (particularly for dental alveoles), a biconvex lens (particularly for dermis, adipose tissue or aponeuroses), a uniconvex lens (particularly for periostium), or else any desired form. In a preferred way, the wall therefore imparts an external form to the device which is a cylinder, trunk-cone, spindle, biconvex or uniconvex lens or any other form suited to the tissue from which one wants to recover cells.

Hollow space is understood to mean a volume defined by the wall of the device, said volume being empty, or containing a central post to avoid unwanted flattening of the wall or comprising a support allowing or promoting the culture or adhesion of cells and/or able to constitute an implant. The shape of said support is adapted by those skilled in the art according to the site of implantation. The support or the internal wall of the device may be functionalized or treated to facilitate colonization or cell growth, for example with biological factors (growth factors and the like) or suitable materials.

In a preferred manner, a device according to the invention has a hollow internal volume comprised between 4 and 3000 mm³, preferably between 4 and 250, even more preferably between 20 and 100 mm³. The device preferably has a length comprised between 2 and 35 mm, preferably between 5 and 15 mm, a width comprised between 2 and 15 mm, preferably between 3 and 6 mm, and a thickness comprised between 1 and 6 mm, even more preferably between 2 and 4 mm.

In a preferred embodiment of the invention, the wall or the device contains a mobile part between a closed position in which the hollow space only communicates with the outside of the device through the pores, and an open position. In open position, the hollow space communicates with the outside of the device by a larger passage allowing the cells or tissues to be recovered.

When the wall of the device contains a mobile part, the latter may be of different nature and form, according to the type of device. For instance, in the case of a device with an elongated shape (for example cylindrical or conical), the mobile part of the wall may be formed by one or more removable ferrules located at the extremities of the device. In the case of a shell-shaped device, for example flattened or lens shaped, the mobile part may be a half of the device, that is to say a half-shell, joined to the other half by a hinge allowing the two half-shells to move apart and come together, a means of locking (or closure) advantageously ensuring that the two half-shells are held against each other. In this respect, some pores may be located to advantageously allow the fixation or the passage of a thread or of any other suitable means (elastic, tongue, etc.) for maintaining the mobile part(s) of the wall in closed position. If the thread is made of a deformable material such as metal, a twist at the exit point may serve as the means of closure. If the thread is non-metallic, single stranded and/or in synthetic material, a knot may be made for the same purpose. The means of closure (or locking) may also have different forms and be produced for instance by moulding with the device as a whole.

The mobile part of the device wall may be designed in any other suitable form. For example, it may be a part of the wall joined to the rest of the device by a mechanical fault line. Thus, a capsule that is torn or cut with a suitable instrument may constitute the mobile part of the wall.

Finally, if the wall does not contain a mobile part, destruction of the device allows it to be opened to recover the cells which proliferated in its internal space.

In a preferred embodiment, the inventive device additionally contains in an advantageous manner a means of traction designed to facilitate removal of the device with the least possible damage to surrounding tissues. The means of traction allows the device to be extracted from the tissues in which it is implanted, on the one hand, and serves to guide the operator to facilitate clearance of the device, on the other hand.

Hence it is possible to use a thin scalpel to clear away tissues adhering to the device, using the means of traction as guide.

The means of traction is therefore generally a protruding piece of the device, such as a guide, strip, thread, tongue, etc. by which to grasp and/or localize the device.

In a first specific embodiment, the means of traction comprises a thread fastened to the device, for example by passage through a pore of the device. The thread may be made of deformable material or not, synthetic or not. Its nature may be metallic, natural (collagen) or synthetic (for example polymer). Its length is advantageously adapted by those skilled in the art so that one free end is accessible after implanting the device in vivo.

The traction thread may therefore be used to remove the device once the in vivo time period is up. An extension of the thread or, more generally, of the means of traction, may be left at a distance from the sampling site and may be stabilized under the epithelium by an additional suture so that it is found easily when it comes time to withdraw the device.

The traction thread may be the same as the thread used to hold the device in closed position.

The means of traction may have a variety of forms. For instance, it may be moulded to the device as a whole, particularly when the latter is made in polymer.

In such case, the inventive device advantageously comprises a wall such as defined hereinabove and a means of traction and/or closure, said components being made of a biocompatible material, preferably selected in the group consisting of titanium, titanium alloys, ceramics, chrome-cobalt, nickel-chrome alloys, or polymers such as polytetrafluoroethane or polycarbonate. The components may be prepared by moulding the whole, or by separate mouldings and assemblies. Moreover, as noted earlier, the means of traction and the means of closure may be one and the same piece, for example a thread.

A particular device of the invention comprises a hollow sleeve constituting the main part of the wall and one or more ferrules each representing or constituting a mobile part of the wall sealing one extremity of the sleeve (see FIG. 1). Each ferrule fits into the hollow sleeve without presenting any protruding parts, so that it does not interfere with insertion of the device in tissues and its subsequent extraction and/or so that it does not sustain an area of inflammation due to damage to the tissue. The mobile part of the wall constituted by the ferrules and sealing the sleeve may for example be hemispherical, conical or flat, depending on the tissue to be sampled. It is preferably hemispherical for a soft and fragile tissue such as adipose tissue, preferably conical for tissues whose components are elongated in shape, such as ligaments and muscles and, preferably, flat with rounded edges for bone tissue.

Another particular device according to the invention comprises a biconvex or uniconvex lens constituting the entire wall, divided into two halves joined by a hinge (see FIG. 6). Advantageously, such device additionally contains an extraction grip joined to one of the two halves. Advantageously, the extraction grip is elastically deformable and at rest passes through a slit in the other half of the lens, thereby preventing the two halves from spreading apart from each other.

The invention furthermore concerns a kit containing a device such as described hereinabove and allowing sampling of cells and/or tissues in vivo in a subject. It further concerns single-use surgical instruments associated with each type of device which may also be supplied in the form of kits. They contain:

a handling tube the internal cross-section of which corresponds to the external cross-section of the device to be implanted, and allowing the device to be conveyed to the desired recipient site with a minimum of handling difficulties and risk of contamination; in an advantageous manner the tube is round for a cylindrical device and flattened for a device presenting the same feature; and/or

a piston fitted to the tube which allows the device to be pushed out of the tube and inserted at the recipient site, said piston preferably comprising a “U” section allowing passage of the means of traction.

The means of traction, if it is a thread, may be fixed to the device beforehand and additionally ensure its closure, if this function is assigned to it, thus allowing it to be recovered easily.

The handling tube and piston may be made in any suitable material, for instance a polymer material, preferably transparent for good visibility during handling, for example polycarbonate.

The device assembly and its handling accessories are preferably provided assembled and sterile in double sachets.

Preparation of Biological Material

The invention provides for the particularly advantageous use of a device such as described hereinabove for in vitro or ex vivo but also in vivo preparation of a tissue, cell culture, transplant or implant. It may be a mammalian tissue and in particular a human tissue. More specifically, it may be a bone, ligament, cartilaginous, muscle, vascular or epithelio-connective tissue for example.

The device or “cell trap” described herein advantageously uses the biological, cellular and molecular dynamic of healing within the tissue of interest.

In particular, the invention provides a protocol by which to obtain, through a suitable surgical trauma, a site where a tissue of interest will go through different phases of healing and different cellular states and to sample the desired cells in the site, under the best possible conditions, that is to say, in vivo, at the chosen time. The implantation of a specific device, also an object of the invention, constitutes the initial surgical trauma and allows “trapping” of active phase cells which colonize the device volume, stimulated by the in situ release of quantities of growth factors.

The healing of a lesion, be it traumatic, infectious, surgical or thermal, follows the same process, provided that the cause has been eliminated and does not sustain chronicity. In the case of a surgical or traumatic lesion, the successive phenomena of healing may be divided into several overlapping phases:

The first phase corresponds to formation of the blood clot. During this step the flow of blood due to the hemorrhage carries fibrin (which creates a network of interlinked fibers) and platelets which, by aggregating, seal the cut vessels and allow formation of a temporary matrix promoting cell migration. These blood platelets are reservoirs of growth factors and cytokines. In fact, once activated, platelets release these molecules which act as the start signal for repair of the lesion. They trigger recruitment of inflammatory cells to the lesion, followed by epithelialization, formation of granulation tissue and angiogenesis.

The early inflammatory phase, which is the second phase, corresponds to the massive arrival of blood polymorphonuclear neutrophils which, attracted by the cytokines, phagocytose foreign particles, debris and bacteria. The neutrophils neutralize these elements by producing enzymes and radical oxygen species which have a destructive effect even with respect to surrounding tissues. Neutrophils also release pro-inflammatory cytokines which attract and activate neighboring fibroblasts and keratinocytes, as well as other immune cells, particularly blood monocytes which become in particular tissue macrophages.

The late inflammatory phase comprises an accumulation of macrophages which continue the work of the neutrophils on the one hand and phagocytose foreign and tissue debris and bacteria on the other hand. They continually synthesize and secrete growth factors and cytokines and therefore continue the production of molecular signals required for the healing process, subsequent to platelets and neutrophils. Macrophages clean the area of all damaged tissues.

Thus, tissue destruction occurs during these inflammatory phases. When an inventive device is implanted, then, it is generally preferable to wait until the end of these initial phases of healing to extract the device and recover the tissue or cells.

In fact, the fibrin clot is subsequently gradually lysed by enzymes (plasmin), allowing the cells, particularly the fibroblasts and keratinocytes, to migrate. Proteases induce resorption of tissue matrix components and particularly collagen, to facilitate cell migration. Finally, other signals exert a chemotactic effect on the cells required for repair.

The granulation tissue phase begins at approximately the fourth day, and is a period of intensive cell proliferation and differentiation, particularly of fibroblasts and new capillaries, with persistence of macrophages and a loose connective tissue.

Granulation tissue thus constitutes a complex reservoir of cytokines with chemotactic, mitogenic and regulatory properties. At this stage there is an important interdependence between cell groups: fibroblasts forming extracellular matrix which serves as a cell scaffold and neovascularization, itself supplying the nutrients needed by the highly activated cells. Fibroblasts also have an autocrine regulation, related at least in part to functional stimulation of cells by tensile constraints within the tissue.

The next phase—tissue remodeling—is a phase where the cells of the tissue in question, which proliferated during the previous phase, differentiate and produce extracellular matrix which repairs and reconstructs the damaged tissue. Fibroblasts are still present during this phase. In the case of bone tissue, osteoblasts are activated at this stage and participate in tissue repair. The tissue gradually matures and a regulation reduces the number of cells when repair is on the verge of completion.

A tissue sampling gives very different results according to whether it is carried out in a mature, intact tissue, or in a recently damaged tissue in the process of healing, and in the second case, according to the time interval after the trauma.

Approximately seven days after the trauma (this period varies according to the tissue and animal species), the granulation tissue phase reaches a maximum, and sampling at this stage will give the largest number of proliferative phase cells.

Approximately fourteen days post-trauma, the matrix formation phase reaches a maximum, and sampling at this stage will give the largest number of active phase differentiated cells.

Any intermediate sampling (or after the initial inflammatory phases) also provides cell populations having altogether advantageous proliferation properties and/or activities. As compared with these different interesting situations, a sampling in a mature intact tissue will provide only few cells, with low activity or quiescent, imprisoned in a dense matrix which they may have difficulty getting out of when placed in culture. The percentage of cells lost in this type of sampling can be high, up to 90-95% of the existing population.

The invention therefore provides a method for preparing a tissue, cell culture or implant by means of a device such as described hereinabove capable of exploiting the natural regeneration capacities of a tissue in the process of healing.

A device according to the invention may thus be implanted in a tissue of interest, preferably healthy, that is to say not having suffered another lesion prior to implantation, so as to obtain a homogeneous regeneration of the tissue. A trauma, such as an incision for example, is thus produced in the tissue of interest from which one seeks to obtain a cell sample then, an inventive device, whose shape is adapted to the tissue of interest, is placed in said tissue, for example by means of a handling tube and piston according to the invention. A suture is then made so as to stabilize the device in the tissue of interest. In a preferred embodiment, the means of traction optionally present is stabilized by an additional suture, at a distance from the device and therefore from the sampling site, and allows easy localization of the latter.

After removing the device (for example in a second surgical procedure), it is opened under aseptic conditions, and its contents are:

either used directly as transplant for another operative site presenting a lesion or tissue deficiency to be treated with the same type of tissue,

or placed in culture or transport medium, for use in a cell therapy method, tissue engineering or research.

If the contents of the device are used as transplant, the latter is advantageously grafted in a site which itself has been prepared in advance in order to be in the tissue remodeling phase with neovascularization which promotes nutrition of the grafted tissue. All the conditions will then be united to obtain very rapid and much more reliable healing.

Another aspect of the invention relates to the advantageous use of a device such as described for preparing a tissue or implant for treating a tissue loss.

Another aspect of the invention concerns a method for repairing a tissue loss or treating a subject, comprising the following steps:

a) producing a trauma in a tissue from which one wants to obtain a cell sample and implanting a device according to the invention in said tissue (these two phases are generally concomitant, the insertion of the device, for example by an incision, constituting the trauma. The insertion of the device typically comprises a suture step to hold the device within the tissue of interest);

b) recovering the device at a determined time at which the cells of the tissue possess the desired properties, for example between days 5 and 21 inclusive, preferably between days 6 and 14 inclusive after insertion; and

c) recovering the cells or tissues in the device and transplanting them, either directly or after culture and/or conditioning, at a recipient site in a subject where a tissue repair is desired or allowing to treat the subject.

In a particular method of repair according to the invention, the device is removed at approximately the fourteenth day, during the remodeling phase. The method is adapted to the repair of bone, ligament, muscle, tendon, connective, vascular tissue and the like.

In a preferred embodiment, the sampled cells are autologous cells. In such case the cells are taken directly from the subject who requires tissue repair. They may also be syngeneic cells collected from an identical twin of the subject to be treated or allogeneic cells from another subject of the same species (related or not), that is to say, another human being in the case of treatment of a human patient.

Other aspects and advantages of the invention will become apparent in the following examples, which are given for purposes of illustration and not by way of limitation, and in the figures as follows:

FIG. 1 is an exploded front view of a cylindrical device according to a first embodiment;

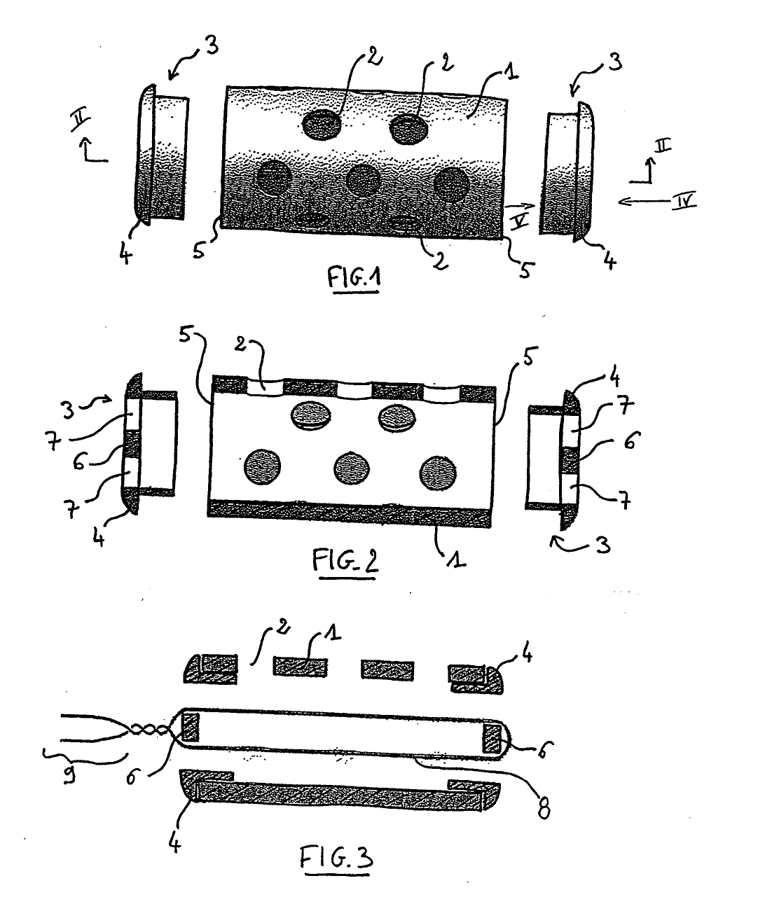

FIG. 2 is a longitudinal sectional view along II-II of FIG. 1;

FIG. 3 is a longitudinal sectional view along II-II of the assembled device;

FIG. 4 is a side view through IV of FIG. 1;

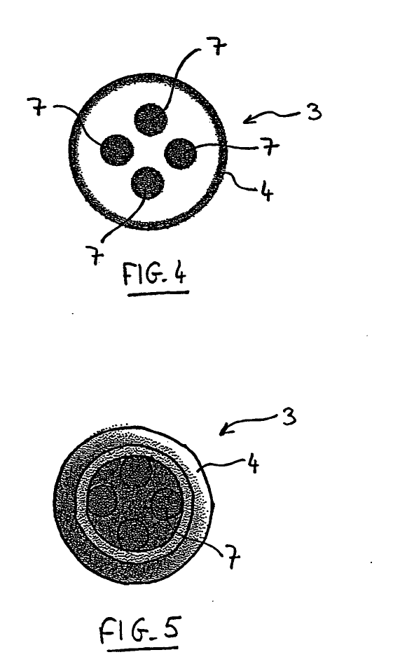

FIG. 5 is a side view through V of FIG. 1 showing the internal face of the ferrule;

FIG. 6 depicts a device according to another embodiment of the invention, in open position;

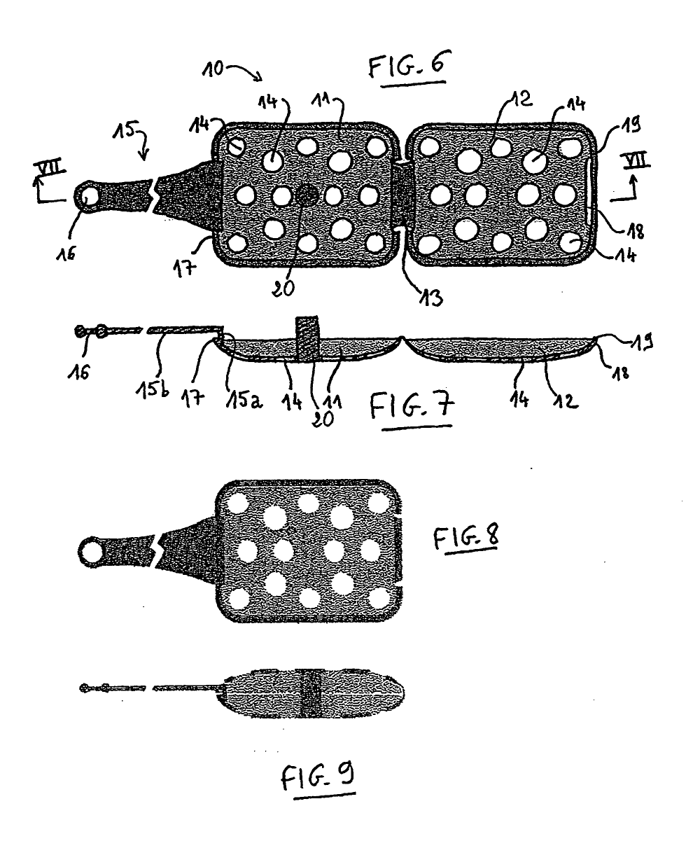

FIG. 7 is a cutaway drawing along VII-VII of FIG. 6;

FIG. 8 is analogous to FIG. 6, with the device in closed position; and

FIG. 9 is a cutaway drawing along IX-IX of FIG. 8.

In the embodiment illustrated in FIG. 1, the device comprises a hollow cylindrical sleeve 1 the lateral wall of which is perforated with pores 2 allowing cells to colonize the internal space of the device.

Two ferrules 3 complete the device. Each ferrule has a diameter slightly less than the internal diameter of the sleeve so that it may be forcefully engaged therein and become immobilized. Each ferrule 3 comprises an end flange 4 which rests on the edge 5 of the sleeve and extends the external surface of the latter, when the ferrule is in position, by its rounded form joining without sharp edges the lateral face of the sleeve to the external face of the ferrule.

Said external face is formed by a transverse diaphragm 6 which constitutes, according to the invention, a mobile part of the device wall.

In the example shown, the diaphragm 6 is joined all in one piece with the rest of the ferrule.

Pores 7 identical or similar to those 2 perforated in the lateral wall of the sleeve are arranged in the transverse diaphragm of each ferrule, as shown in FIGS. 4 and 5.

It can be seen that the mobile part of the wall formed by the diaphragms 6 can adopt a closed position, in which the ferrules are engaged on the sleeve. The internal space of the device then communicates with the outside only through pores 2,7, and in an open position in which the ferrules are removed. In this latter case, the internal space of the device is open to the outside by a passage the size of its cross-section.

FIG. 3 illustrates a suture thread 8 forming a loop passing through the pores 7 of the ferrules. By being twisted at its exit point from the device, the suture thread remains taut between the two ferrules 3 and thus holds said ferrules joined to sleeve 1.

The extension 9 of suture thread 8 serves as a traction thread to extract the device after the trapping period.

In a variant, the thread may be knotted instead of twisted.

In the embodiment shown in FIGS. 6 to 9, the device has a generally flat shell shape 10 formed by two half-shells 11, 12 joined by a flexible hinge 13.

The wall of each half-shell is perforated with colonization pores 14.

Each half-shell is mobile relative to the other one. By agreement, half-shell 12 is considered to constitute the mobile part of the device wall.

In accordance with the invention, the half-shell 12 may adopt a closed position, represented in FIGS. 8 and 9, in which the internal space of the device only communicates with the outside through the pores 14, and an open position, depicted in FIG. 7, in which the inside communicates with the outside over its entire cross-section, shown in its principal plane.

In its principal plane, that in FIG. 6, the shell has a globally rectangular cross-section. In a perpendicular plane, particularly that of FIGS. 7 and 9, the device has a cone-shaped cross-section.

One half-shell 11 contains, opposite hinge 13, an extraction grip 15 terminating in a fixation hole 16 through which said grip may be sutured sub-epithelially for later recovery.

As shown in FIGS. 7 and 9, the extraction grip 15 is composed of two arms, one 15a which is short, the other 15b which is long, perpendicular to each other. The short arm 15a arises from the inside of the half-shell 11 and comes up perpendicularly to the principal plane of the half-shell, along its edge 17 opposite hinge 13. The long arm 15b is located a short distance from the edge of the half-shell and forms a right angle with the short arm 15a.

The other half-shell contains a slit 18 along the edge 19 opposite hinge 13, the length of which is slightly greater than the width of the extraction grip 15.

The extraction grip, like the rest of the device moreover, is made of supple plastic material. It is sufficiently deformable to be able to be elastically curved on itself so that its free end, with fixation hole 16, lies along the path of slit 18 as the two half-shells come together by rotation around hinge 13. When the grip meets the slit, it engages therein and the device continues to close until the two half-shells are held firmly against each other. Just before closure of the device, the slit clears the right angle formed by the two arms of the grip. The extractop, grip then resumes its resting position to hold the device in closed position, as illustrated in FIG. 9.

Subsequent opening of the device to remove the cells that have colonized therein is accomplished by a reverse motion, facilitating the spreading apart of the two half-shells through a deforming action on the grip.

To prevent the device from being crushed when in closed position, one 11 of the two half-shells contains a central post 20 which rests on the internal face of the other half-shell, as also seen in FIG. 9.

### EXAMPLES

### Example 1

### Sampling of Bone Cells

Entry was through an incision of at least 4 centimeters in an accessible bone, for example, the edentate crest of a maxilla or the anteromedial border of the tibia. The musosa-periostium was then detached. At 1 cm from an extremity, the bone was gradually drilled, from 2 to 5 mm in diameter, per mm, with irrigation, and for a depth of 10 mm, to fit the dimensions of the “cell trap”. The latter was placed with the “trap holder” in the alveole so prepared, and the extraction thread was laid against the surface of the bone up to the other end of the incision. Cutaneous or mucosal sutures were made to fix an end loop of the extraction thread of the trap subcutaneously or in the mucosa.

A guided tissue regeneration membrane can optionally be placed above the alveolar opening, trap and thread, against the bone surface, extending at least 5 mm over each side of the alveole so as to prevent invasion of soft tissues.

The different phases of healing of the bone tissue then take place, with release of growth factors and cellular activations.

The “trap” can be removed 14 days after implantation to extract therefrom a cell population derived from the surrounding bone tissue and differentiated into osteoblasts.

Said cells may then be cultured in vitro, in a much more favorable manner than if one had to wait for cells to migrate from bone tissue explants.

The tissue may also be grafted at a site so requiring it.

### Example 2

### Sampling of Tendon or Ligament Fibroblasts

A small (length: 8 mm, diameter: 3 mm), cylindrical trap with conical ends was used. It was implanted in a tendon or ligament after incision through the skin and gradual dissection.

It was stabilized within the ligament by a suture. The traction thread was also stabilized and fixed subcutaneously with another suture. The skin suture was then made.

The cell sample may be collected by removing the “cell trap” after 14 days, so as to collect the greatest number of differentiated cells which colonized the inside of the trap. Fairly large openings (18) in the ferrules of the trap may promote functional stimulation therein and thus enhance cellular differentiation.

Once the trap is removed, the sutures are redone.

As with bone, the cells so obtained do not have to detach from a dense matrix before being able to be cultured, which considerably accelerates the in vitro culture.

Said cells may also be placed against a ligamental lesion in view of its repair.

### Example 3

### Sampling of Cells from Periodontal Ligament

Following a tooth extraction, a trunk-cone “trap” may be placed in the dental alveole to collect cells of the periodontal ligament. In fact, said cells proliferate rapidly at the surface of the alveolar cavity after any extraction, provided that the cavity was not dredged.

After one week, said cells can differentiate into osteoblasts. The time during which the trap is left in place determines the nature of the cells harvested. After implantation of the trap in the alveole of an extracted tooth, a mucosal flap should cover and seal the alveole during the entire time.

The principles of suture are the same and the traction thread is brought to about 3 cm from the “trap”.

### Example 4

### Sampling of Periostial Cells

A flat uniconvex “trap” was preferred. The sampling site was an accessible bone surface (tibia, palate). An entry incision followed by detachment of periostium over 4 cm were necessary to glide the “trap” into the “pocket” so prepared. It is important to have good contact with the bone. The planes (aponeurosis and skin) were resutured by stabilizing the end loop of the extraction thread to the other end of the incision.

A period of 14 to 21 days was necessary to obtain a sufficient osteoblast population. The “trap” was then removed using the extraction thread as guide.

1-24. (canceled).

25. Device for providing implants, cells or tissues, comprising a wall in biocompatible material defining a hollow space, said wall containing pores (2, 7, 14) allowing cells to colonize said hollow space.

26. Device according to claim 25, wherein the ratio between the overall area of the pores (2, 7, 14) and the overall external area of the wall is less than 0.5, particularly comprised between 0.1 and 0.5, typically between 0.1 and 0.4.

27. Device according to claim 25, wherein the wall contains a mobile part (3, 12) between a closed position in which the hollow space communicates with the outside of the device only through the pores (2, 7, 14) and an open position.

28. Device according to claim 25, wherein it comprises a means of traction (9, 15).

29. Device according to claim 25, wherein the wall imparts to the device an external form which is cylindrical, trunk-cone, spindle, biconvex or uniconvex or any other form adapted to the tissue from which one wants to sample cells.

30. Device according to claim 25, wherein the wall and the means of traction are made of a biocompatible material based on titanium, titanium alloys, ceramics, chrome-cobalt, nickel-chrome alloys or polymers such as polytetrafluoroethane or polycarbonate.

31. Device according to claim 25, wherein the pores (2, 7, 14) are round, elongated and/or oval and/or any other form adapted to the tissue.

32. Device according to claim 25, wherein it has an internal hollow volume comprised between 4 and 3000 mm3, preferably between 4 and 250, even more preferably between 20 and 100 mm3 for a length comprised between 2 and 35 mm, preferably between 5 and 15 mm, a width comprised between 2 and 15 mm, preferably between 3 and 6 mm, and a thickness comprised between 1 and 6 mm, even more preferably, between 2 and 4 mm.

33. Device according to claim 25, wherein some pores (7) are arranged so as to permit fixation of the means of traction.

34. Device according to claim 25, wherein the means of traction is a thread (8) which holds the mobile part of the wall (6) in closed position by passing through several pores (7).

35. Device according to claim 25, wherein the means of traction is designed to be left at a distance from the sampling site and stabilized sub-epithelially by an additional suture for easy localization.

36. Device according to claim 25, wherein it comprises a hollow sleeve (1) constituting the main part of the wall, and one or more ferrules (3) each constituting a mobile part of the wall (6) sealing one extremity of the sleeve.

37. Device according to claim 25, wherein it comprises a hollow sleeve (1) constituting the main part of the wall, and one or more ferrules (3) each constituting a mobile part of the wall (6) sealing one extremity of the sleeve and wherein each ferrule (3) fits into the hollow sleeve (1) so that there are no protruding parts, so as not to interfere with insertion of the device in the tissues and its extraction.

38. Device according to claim 25, wherein it comprises a hollow sleeve (1) constituting the main part of the wall, and one or more ferrules (3) each constituting a mobile part of the wall (6) sealing one extremity of the sleeve and wherein the mobile part of the wall (6) constituted by the ferrules is hemispherical for a soft and fragile tissue such as adipose tissue, conical for tissues whose constitutive components have an elongated shape, such as ligaments and muscles, and flat with rounded edges for bone tissue.

39. Device according to claim 25, wherein it comprises a biconvex or uniconvex lens constituting the entire wall, divided into two halves (11, 12) which are joined by a hinge (13) and, preferably, an extraction grip (15) joined to one of the two halves.

40. Device according to claim 25, wherein it comprises a biconvex or uniconvex lens constituting the entire wall, divided into two halves (11, 12) which are joined by a hinge (13) and, preferably, an extraction grip (15) joined to one of the two halves and wherein said extraction grip (15) is elastically deformable and when at rest crosses a slit (18) in the other half thus preventing the two halves from spreading apart from one another.

41. Method for preparing in vitro or ex vivo a tissue, cell culture or implant, wherein a device according to claim 25 is used.

42. Method for preparing a kit for sampling cells and/or tissue in vivo in a subject, wherein a device according to claim 25 is used.

43. Method for preparing a composition for carrying out a method of therapeutic or surgical treatment of the human body, in particular a cell therapy method, wherein a device according to claim 25 is implemented.

44. Method for preparing a mammalian tissue and in particular a human tissue, wherein a device according to claim 25 is used.

45. Method according to claim 44, wherein the mammalian tissue is a bone, ligament, cartilaginous, muscle, vascular or epithelio-connective tissue.

46. Kit wherein it contains a device according to claim 25.

47. Kit according to claim 46, wherein, in addition to the device, it contains:
(i) a handling tube the internal cross-section of which corresponds to the external cross-section of the device to be inserted and allowing said device to be conveyed to a recipient site, and/or
(ii) a piston fitted to the tube (i) which allows said device to be pushed out of the tube and inserted in the recipient site, said piston preferably having a “U” section allowing passage of the means of traction.

48. Kit according to claim 46, wherein, in addition to the device, it contains:
(i) a handling tube the internal cross-section of which corresponds to the external cross-section of the device to be inserted and allowing said device to be conveyed to a recipient site, and/or
(ii) a piston fitted to the tube (i) which allows said device to be pushed out of the tube and inserted in the recipient site, said piston preferably having a “U” section allowing passage of the means of traction,
and wherein the handling tube and piston are in polymer material, preferably transparent, for example polycarbonate.
